(19) United States
(12) Patent Application Publication
(10) Pub. No.: US 2009/0175463 A1
(43) Pub. Date: Jul. 9, 2009

(54) NOISE-CANCELING SOUND PLAYING STRUCTURE

(75) Inventors: Ping-Yu Lee, Taoyuan City (TW); Yi-Wei Kuo, Taoyuan City (TW); Hsin-Yuan Chiang, Taoyuan City (TW)

Correspondence Address: SCHMEISER, OLSEN & WATTS, 22 CENTURY HILL DRIVE, SUITE 302, LATHAM, NY, 12110, US

(73) Assignee: FORTUNE GRAND TECHNOLOGY INC., Taoyuan City (TW)

(21) Appl. No.: 11/970,554

(22) Filed: Jan. 8, 2008

(51) Int. Cl. A61F 11/06; H04R 5/00

(52) U.S. Cl. 381/71.6; 381/71.1; 381/72

(57) ABSTRACT

A noise-canceling sound playing structure includes a noise canceling mechanism having at least one filtering and phase-reversing unit, an amplifier, and a power supply unit; and a sound output mechanism having at least one enclosure. The enclosure has a first speaker arranged therein and connected to the amplifier, a second speaker arranged therein and connected to an external sound player, and a microphone mounted thereon and connected to the filtering and phase-reversing unit. A chamber is defined in the enclosure between the first and second speakers and the microphone. External noise signals are picked up by the microphone and sent to the noise canceling mechanism for processing at the filtering and phase-reversing unit and the amplifier to thereby produce phase-reversed signals, which are sent to the first speaker to cancel out external noise without being mixed with the musical sound transmitted from the external sound player to the second speaker.

### FIELD OF THE INVENTION

The present invention relates to a noise-canceling sound playing structure, and more particularly to a noise-canceling sound playing structure that does not mix musical sound with noise signal to keep the fidelity of musical sound being played, and has fewer electronic components to enable reduced overall volume, power consumption, and manufacturing cost thereof.

### BACKGROUND OF THE INVENTION

A conventional noise-canceling audio device is achieved generally by applying the active noise canceling (ANC) technology to stereo headphones. FIG. 1 is a block diagram of a pair of conventional noise-canceling headphones, which includes two sets of speaker 4 and microphone 5, and an ANC module 6. Each of the two headphones has one speaker 4 and one microphone 5 parallelly arranged therein, and the ANC module 6 includes two independently operable sets of filtering and phase-reversing units 61 and amplifier 62, and a power supply unit 63.

The two microphones 5 for the pair of headphones separately pick up noise signals in external environment, and send the noise signals to the ANC module 6 for processing at the two filtering and phase-reversing units 61, so that noises having frequencies that are not perceptible by human ears are filtered out first, and noises that are perceptible by human ears are then subjected to phase reversal by 180 degrees. The phase-reversed noise signals are then amplified by the amplifiers 62 and mixed with the musical signals transmitted from an external music player 7 to the headphones. Finally, the phase-reversed noises are transmitted by the two speakers 4 at the same time to cancel the external noises and sent to the user's ears along with the musical sound.

The above-described conventional pair of noise-canceling headphones has the following disadvantages:
(a) Since two independent amplifiers 62 are included for amplifying the noise signals that have been filtered and phase reversed at the two independent filtering and phase-reversing units 61, the ANC module 6 has a circuit board of a relatively large volume that could not be reduced in size. Moreover, the number of electronic components in the ANC module 6 is large, which causes not only high manufacturing cost but also increased power consumption to adversely affect the duration of use of the headphones.
(b) The phase-reversed noises are mixed with the musical sound, and the mixed sound is sent to the speakers 4 for playing. With the phase-reversed noises mixed therewith, the musical sound within some frequency bands tends to lose fidelity.

### SUMMARY OF THE INVENTION

It is therefore a primary object of the present invention to provide a noise-canceling sound playing structure that does not mix musical sound with noise signal to keep the fidelity of musical sound being played, and has fewer electronic components to enable reduced overall volume, power consumption, and manufacturing cost thereof.

To achieve the above and other objects, the noise-canceling sound playing structure according to a preferred embodiment of the present invention includes a noise canceling mechanism having at least one filtering and phase-reversing unit, an amplifier, and a power supply unit; and a sound output mechanism having at least one enclosure. The enclosure is arranged therein a first speaker electrically connect to the amplifier, a second speaker electrically connected to an external sound player, and a microphone mounted on the enclosure and connected to the filtering and phase-reversing unit. A chamber is defined in the enclosure between the first and second speakers and the microphone.

External noise signals are picked up by the microphone and sent to the noise canceling mechanism for processing at the filtering and phase-reversing unit and the amplifier to produce phase-reversed signals, which are sent to the first speaker to cancel out external noise signals without being mixed with the musical sound transmitted from the external sound player to the second speaker.

With only one amplifier provided in the noise canceling mechanism, the noise-canceling sound playing structure of the present invention has a reduced number of electronic components to thereby enable reduced circuit board size and overall volume, power consumption, and manufacturing cost thereof.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a block diagram of a conventional pair of noise-canceling headphones;

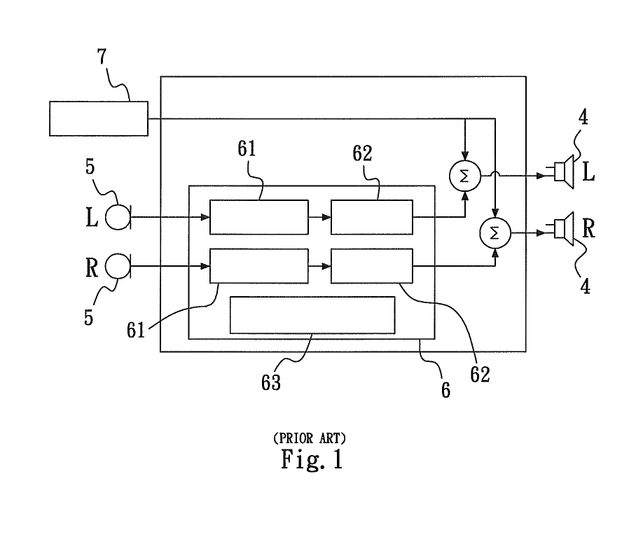

FIG. 2 is a block diagram of a noise-canceling sound playing structure according to a preferred embodiment of the present invention;

FIG. 3 is a sectional view of a sound output mechanism adopted in the noise-canceling sound playing structure in the preferred embodiment of the present invention;

FIG. 4 is a perspective view showing an example of implementation of the present invention;

FIG. 5 is a sectional view showing another embodiment of the sound output mechanism adopted in the noise-canceling sound playing structure of the present invention; and

FIG. 6 is a sectional view showing a further embodiment of the sound output mechanism adopted in the noise-canceling sound playing structure of the present invention.

### DETAILED DESCRIPTION OF THE PREFERRED EMBODIMENTS

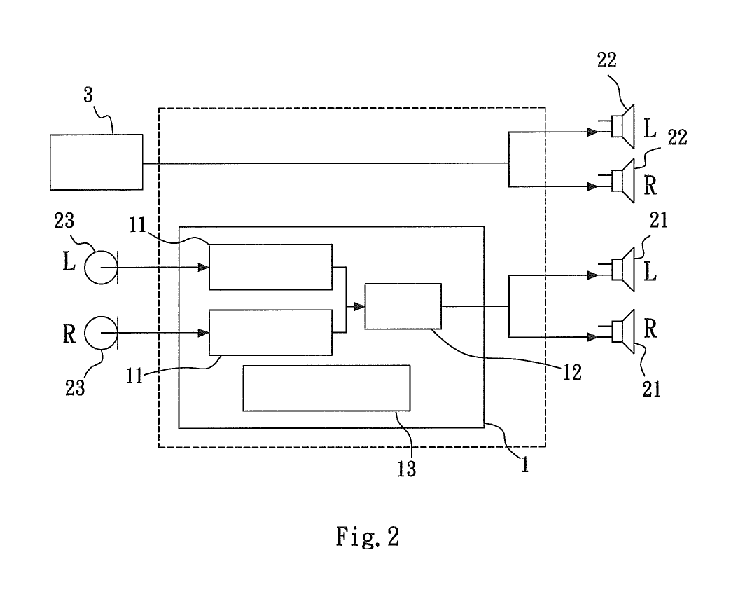

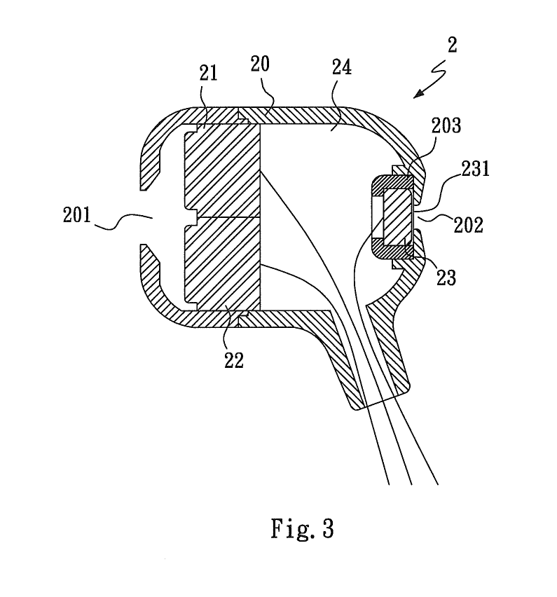

A noise-canceling sound playing structure according to a preferred embodiment of the present invention includes a noise canceling mechanism 1 and a sound output mechanism 2. The sound output mechanism 2 may be a single-ear headphone or a dual-ear headphone set. In the illustrated preferred embodiment of the present invention, based on which the present invention is described, the sound output mechanism 2 is a dual-ear headphone set. Please refer to FIG. 2 that is a block diagram of the preferred embodiment of the present invention, and to FIG. 3 that is a sectional view of a sound output mechanism 2 adopted in the preferred embodiment of the present invention.

The noise canceling mechanism 1 includes two filtering and phase-reversing units 11, an amplifier 12 connected to the two filtering and phase-reversing units 11, and a power supply unit 13 connected to the filtering and phase-reversing units 11 and the amplifier 12.

In the preferred embodiment of the present invention, the sound output mechanism 2 includes two enclosures 20, in each of which a first speaker 21 is arranged near a first end wall of the enclosure 20 and electrically connected to the amplifier 12, a second speaker 22 is also arranged near the first end wall of the enclosure 20 and electrically connected to an external sound player 3, and a microphone 23 mounted to a predetermined position on the enclosure 20 and electrically connected to a corresponding one of the two filtering and phase-reversing units 11. A chamber 24 is defined in the enclosure 20 between the first and second speakers 21, 22 and the microphone 23.

The enclosure 20 is provided at the first end wall having the first and second speakers 21, 22 arranged thereat with a sound releasing section 201, and at an opposite second end wall with a sound hole 202. The enclosure 20 is internally formed of a locating section 203 corresponding to the sound hole 202 for receiving the microphone 23 therein, so that the microphone 23 is isolated from the first and second speakers 21, 22 and the chamber 24 is not commonly used by the speakers 21, 22 and the microphone 23.

The microphone 23 has a pick-up end 231 corresponding to the sound hole 202 for picking up external noise signals. The pick-up end 231 may be adjoined to the second end wall of the enclosure 20 or adjoined to the chamber 24 in the enclosure 20. Alternatively, the microphone 23 may be located at a position (not shown) in the vicinity of the first and second speakers 21, 22.

The above-mentioned configuration is only a preferred embodiment of the present invention. It is understood, in actual implementation of the present invention, the first speaker 21, the second speaker 22, and the microphone 23 may be associated with the enclosure 20 at other different positions according to different conditions of use without being limited to the arrangement described in the preferred embodiment.

In implementing the present invention, the noise canceling mechanism 1 may be arranged at different positions according to actual need. For example, the noise canceling mechanism 1 may be arranged in the enclosure 20 of the sound output mechanism 2, in the external sound player 3, or in a control mechanism 30 which is connected to the external sound player 3

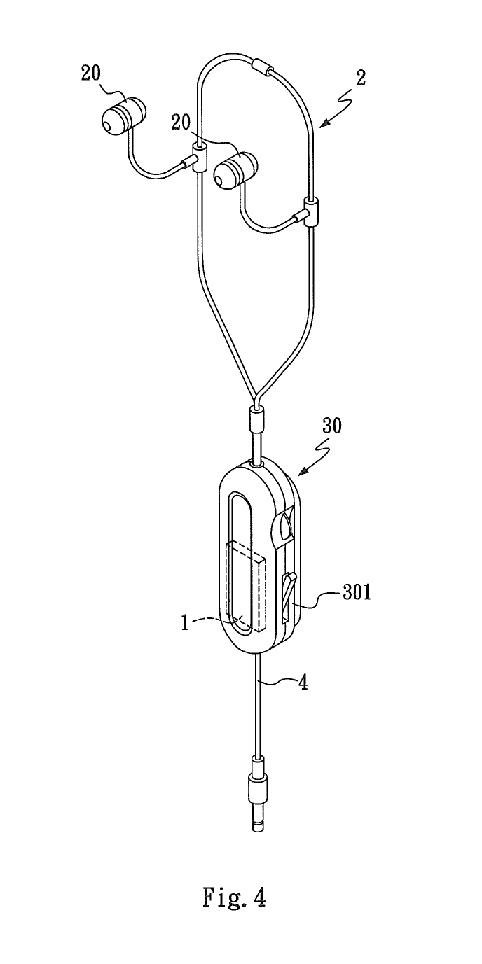

Please refer to FIG. 4. The control mechanism 30 connected to the external sound player 3 includes a switch 301 electrically connected to the power supply unit 13 of the noise canceling mechanism 1 arranged in the control mechanism 30 for turning on the power supply unit 13 and actuating the noise canceling mechanism 1. The control mechanism 30 is also electrically connected to the sound output mechanism 2.

When using the noise-canceling sound playing structure of the present invention, musical sound from the external sound player 3 is directly transmitted via a sound line 4 to the second speaker 22 in each enclosure 20. And, when the power supply unit 13 is turned on, the microphone 23 in each enclosure 20 automatically picks up external noises at the pick-up end 231. External noises picked up by the microphones 23 at both enclosures 20 are transmitted to the noise canceling mechanism 1, and processed at the filtering and phase-reversing units 11 and the amplifier 12 to produce phase-reversed signals. The phase-reversed signals are sent to the first speakers 21 to produce phase-reversed noise wave, so as to cancel out the noises come from external environment. Therefore, a complete noise-canceling audio system is established for the noise-canceling sound playing structure of the present invention.

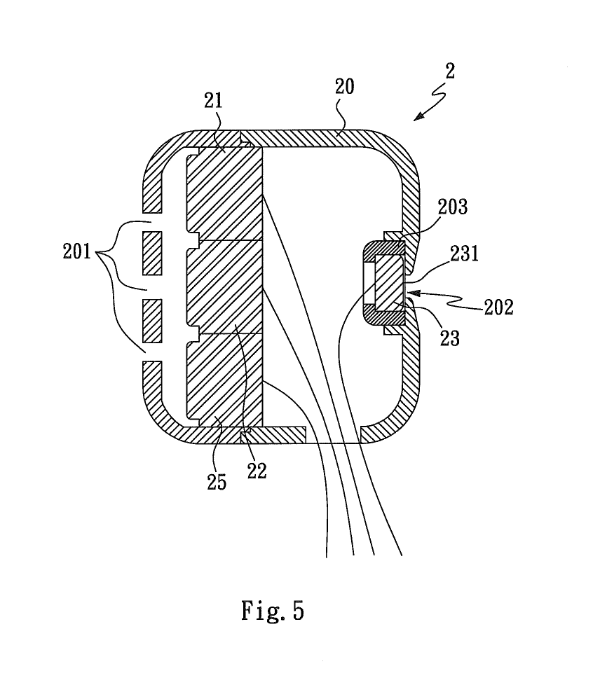

In another embodiment of the sound output mechanism 2 as shown in FIG. 5, at least a third speaker 25 may be further arranged near the first end wall of each enclosure 20, and multiple sound releasing sections 201 are correspondingly formed on the first end wall of the enclosure 20. With the third speaker 25, the sound playing structure of the present invention is equipped with a multi-sound-channel noise canceling mechanism, and may be used in different conditions.

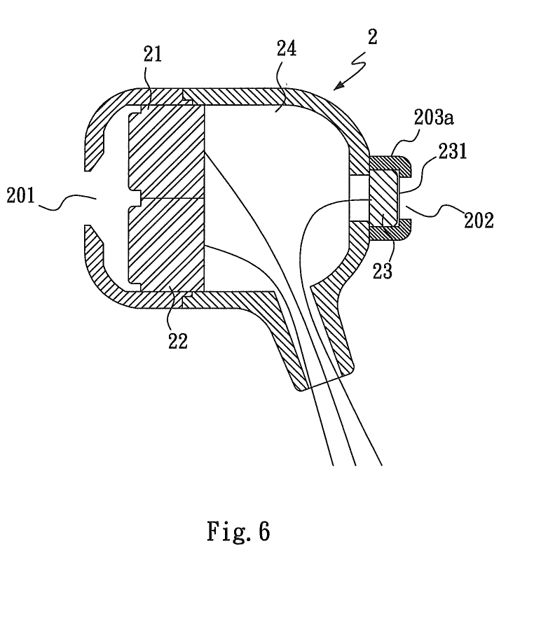

FIG. 6 shows a further embodiment of the noise canceling mechanism 2. In this further embodiment, each enclosure 20 of the noise canceling mechanism 2 has a locating section 203a formed at an outer side thereof for receiving the microphone 23 therein, so that the microphone 23 is isolated from the first and second speakers 21, 22, and the chamber 24 is not commonly used by the microphone 23 and the first and second speakers 21, 22. Again, the microphone 23 has a pick-up end 231 corresponding to a sound hole 202 on the locating section 203a for picking up external noise signals. With the microphone 23 located outside the enclosure 20 as in the further embodiment of the noise canceling mechanism 2, the present invention can still pick up external noise signals and achieve the same noise canceling effect.

In the sound playing structure of the present invention, the first speakers 21 are used to play phase-reversed noise wave, and the second speakers 22 are used to play the musical sound alone. Therefore, the problem of having a musical sound with poor quality possibly caused by mixture of the musical sound with the phase-reversed noise wave as would be found in the prior art is effectively overcome in the present invention. Moreover, the noise canceling mechanism 1 of the present invention includes only one amplifier 12, enabling the present invention to use fewer electronic components and has volume-reduced circuit board and lowered manufacturing cost.

In conclusion, the noise-canceling sound playing structure of the present invention has the following advantages:
a. Fewer electronic components are used to thereby reduce the manufacturing cost of the noise-canceling sound playing structure.
b. Lower power consumption is needed to thereby enable extended duration of use.
c. The musical sound is no longer mixed with the phase-reversed noise signals and can therefore be transmitted to the speakers without distortion, and a user may listen and enjoy music without being disturbed by noises from external environment.
d. Volume-reduced circuit board enables the sound playing structure to meet the trend of compact digital sound player.

With the above arrangements, the noise-canceling sound playing structure of the present invention is improved and more practical for use.

What is claimed is:

1. A noise-canceling sound playing structure, comprising:
a noise canceling mechanism including at least one filtering and phase-reversing unit, an amplifier connected to the filtering and phase-reversing unit, and a power supply unit connected to the filtering and phase-reversing unit and the amplifier; and
a sound output mechanism including at least one enclosure, a first speaker arranged in each enclosure and electrically connected to the amplifier of the noise canceling mechanism, a second speaker arranged in each enclosure and electrically connected to an external sound player, and a microphone mounted on each enclosure and electrically connected to one corresponding filtering and phase-reversing unit in the noise canceling mechanism.

2. The noise-canceling sound playing structure as claimed in claim 1, wherein the noise canceling mechanism is located in the enclosure of the sound output mechanism.

3. The noise-canceling sound playing structure as claimed in claim 1, wherein the noise canceling mechanism is associated with the external sound player.

4. The noise-canceling sound playing structure as claimed in claim 1, wherein the sound output mechanism is a single-ear headphone.

5. The noise-canceling sound playing structure as claimed in claim 1, wherein the sound output mechanism is a dual-ear headphone set.

6. The noise-canceling sound playing structure as claimed in claim 1, wherein each enclosure includes a sound releasing section located near the first and the second speaker, and a locating section formed inside the enclosure for receiving the microphone therein.

7. The noise-canceling sound playing structure as claimed in claim 1, wherein the enclosure includes a sound releasing section located near the first and the second speaker, and a locating section formed outside the enclosure for receiving the microphone therein.

8. The noise-canceling sound playing structure as claimed in claim 1, wherein each microphone has a pick-up end located in the enclosure.

9. The noise-canceling sound playing structure as claimed in claim 1, wherein each microphone has a pick-up end located outside the enclosure.

10. The noise-canceling sound playing structure as claimed in claim 1, wherein each microphone has a pick-up end, and the microphone is located in the vicinity of the first and second speakers.

11. The noise-canceling sound playing structure as claimed in claim 1, wherein the sound output mechanism further includes at least a third speaker in each enclosure.
